(19) United States
(12) Patent Application Publication
(10) Pub. No.: US 2022/0023650 A1
(43) Pub. Date: Jan. 27, 2022

(54) PORTABLE RESUSCITATION DEVICE

(71) Applicant: MedTech LLC, New Rochelle, NY (US)

(72) Inventors: Jonathan Perl, Los Angeles, CA (US); Joseph M. Barrett, Tempe, AZ (US); Daniel W. Allen, Mesa, AZ (US); Joseph Pepe Elijio Velasquez, Tempe, AZ (US)

(21) Appl. No.: 16/940,366

(22) Filed: Jul. 27, 2020

(51) Int. Cl. A61N 1/39; A61M 16/00; A61M 16/10

(52) U.S. Cl. CPC A61N 1/39044; A61N 1/3975; A61N 1/3993; A61N 1/3968; A61M 2205/054; A61M 16/1005; A61M 2205/8206; A61M 2205/8262; A61M 2202/0208; A61M 16/0057

(57) ABSTRACT

A resuscitation device includes a first resuscitation unit configured to perform a first resuscitation function and having front and back sides. A second resuscitation unit is configured to perform a second resuscitation function, which is different from the first resuscitation function, and includes front and rear sides. The first and second resuscitation units are configured so as to be assembled together as a single assembly such that the first and second resuscitation units can be carried conjointly for selectively performing at least one of the first and second functions. An adaptor is also provided for connecting a defibrillator to a plurality of different electrode pads.

### FIELD OF THE INVENTION

The present invention relates to portable resuscitation devices.

### BACKGROUND OF THE INVENTION

Various resuscitation devices have been developed in the past (see, e.g., U.S. Pat. Nos. 7,672,720, 8,078,288 and 8,260,413). Improved resuscitation devices are necessary in providing critical medical care to patients in life-threatening situations, such as patients having a cardiac arrest.

### SUMMARY OF THE INVENTION

In one embodiment, a resuscitation device includes a first resuscitation unit configured to perform a first resuscitation function and having front and back sides. A second resuscitation unit is configured to perform a second resuscitation function, which is different from the first resuscitation function, and includes front and rear sides. The first and second resuscitation units are configured so as to be assembled together as a single assembly such that the first and second resuscitation units can be carried conjointly for selectively performing at least one of the first and second functions.

In one embodiment, the first and second resuscitation units are configured to be arranged in a back-to-back fashion. In one embodiment, the back side of the first resuscitation unit is configured to be removably attached to the back side of the second resuscitation unit.

In one embodiment, the first resuscitation unit includes a first handle, and the second resuscitation unit includes a second handle. The first and second handles are configured such that when the first and second resuscitation units are assembled together, the first and second handles abut one another so as to form a single combined handle for conjointly carrying the first and second resuscitation units.

In one embodiment, at least one storage pouch is configured to be removably attached to one side of the first resuscitation unit and to one side of the second resuscitation unit when the first and second resuscitation units are assembled together as the single assembly. In one embodiment, the at least one storage pouch is configured to inhibit the first and second resuscitation units from becoming detached from each other when the at least one storage pouch is attached to the one side of the first resuscitation unit and the one side of the second resuscitation unit.

In one embodiment, a third resuscitation unit is configured to perform a third resuscitation function different from the first and second resuscitation functions. Each of the first, second and third resuscitation units is configured so as to be assembled together with another one of the first, second and third resuscitation units as a single assembly such that at least two of the first, second and third resuscitation units can be carried conjointly for selectively performing their respective ones of the first, second and third resuscitation functions.

In one embodiment, each of the first and second resuscitation units includes one of an automatic external defibrillator, a ventilator and an oxygenator.

In one embodiment, each of the first and second resuscitation units includes at least one connector configured for connection to an external device having a battery. In one embodiment, each of the first and second resuscitation units contains circuitry for operating a corresponding one of the first and second resuscitation units via electricity received from the battery of the external device.

In one embodiment, each of the first and second resuscitation units includes a connector configured to be connected for connection to an external power supply including at least one of a DC power supply, an AC power supply and a solar power supply.

In one embodiment, at least one of the first and second resuscitation units includes a video conference feature which is equipped with a camera and a display panel mounted on a housing of a corresponding one of the first and second resuscitation units.

In one embodiment, the resuscitation device includes a basket having a first compartment configured to receive the first resuscitation unit and a second compartment configured to receive the second resuscitation unit. In one embodiment, the basket includes at least one pouch for storing supplies and equipment.

In one embodiment, one of the first and second resuscitation units includes a bay. The other one of the first and second resuscitation units is configured to be received in the bay so as to form the single assembly.

In one embodiment, one of the first and second resuscitation units includes a ledge. The other one of the first and second resuscitation units is configured to be received on the ledge so as to form the single assembly.

In one embodiment, a portable defibrillator system includes a portable defibrillator configured for delivering an electrical shock to a patient. The portable defibrillator includes a first connector specifically configured for connection only with a first set of preconfigured electrode pad connectors. The portable defibrillator system also includes an adaptor having a second connector, which is specifically configured for connection with the first connector of the defibrillator, and a plurality of third connectors, at least one of which is specifically configured for connection only with a second set of preconfigured electrode pad connectors, which are different from the first set of preconfigured electrode pad connectors. The second connector is electrically connected to the plurality of third connectors such that the first connector of the defibrillator can be electrically connected to the second set of preconfigured electrode pad connectors via the adaptor.

In one embodiment, each of the plurality of third connectors is specifically configured for connection only with one set of preconfigured electrode pad connectors.

In one embodiment, the portable defibrillator includes at least one connector configured for connection to an external device having a battery. In one embodiment, the portable defibrillator includes circuitry for operating the portable defibrillator via electricity received from the battery of the external device.

In one embodiment, the portable defibrillator includes a power connector configured for connection with an external power supply including at least one of a DC power supply, an AC power supply and a solar power supply.

In one embodiment, an adaptor is provided for a portable defibrillator configured for delivering an electrical shock to a patient. The portable defibrillator includes a connector specifically configured for connection only with a first set of preconfigured electrode pad connectors. The adaptor includes a first connector, which is specifically configured for connection with the connector of the defibrillator, and a plurality of second connectors, at least one of which is specifically configured for connection only with a second set of preconfigured electrode pad connectors, which are different from the first set of preconfigured electrode pad connectors. The first connector is electrically connected to the plurality of second connectors such that the connector of the defibrillator can be electrically connected to the second set of preconfigured electrode pad connectors via the adaptor.

### BRIEF DESCRIPTION OF THE DRAWINGS

In the drawing figures, which are not to scale, and where like reference numerals indicate like elements throughout the several views:

FIG. 1 is a perspective view of a resuscitation device constructed in accordance with an embodiment, the resuscitation device including defibrillator and ventilator units attached to each other to form a single portable assembly;

FIG. 2 is another perspective view of the resuscitation device shown in FIG. 1;

FIG. 3 is a view similar to FIG. 1, except that the resuscitation device is shown without storage pouches;

FIG. 4 is a perspective view of the resuscitation device shown in FIG. 3, the defibrillator and ventilator units shown in their disassembled state;

FIG. 5 is a front view of the resuscitation device shown in FIG. 3;

FIG. 6 is a rear view of the resuscitation device shown in FIG. 3;

FIG. 7 is a top view of the resuscitation device shown in FIG. 3;

FIG. 8 is a bottom view of the resuscitation device shown in FIG. 3;

FIG. 9 is a side view of the resuscitation device shown in FIG. 3;

FIG. 10 is another side view of the resuscitation device shown in FIG. 3;

FIG. 11 is a view of a rear side of the defibrillator unit shown in FIG. 3;

FIG. 12 is a view of a rear side of the ventilator unit shown in FIG. 3;

FIG. 13 is a perspective view of the resuscitation device shown in FIG. 3, illustrating a solar panel and a mobile device for use in conjunction with the resuscitation device in accordance with an embodiment;

FIGS. 14 and 15 are sectional views of the resuscitation device shown in FIG. 3, illustrating an adapter constructed in accordance with an embodiment;

FIG. 16 is a side view of the adapter shown in FIGS. 14 and 15;

FIG. 17 is a front view of the adapter shown in FIGS. 14 and 15;

FIG. 18 is a top view of the adapter shown in FIGS. 14 and 15;

FIG. 19 is a perspective view of an adapter constructed in accordance with one embodiment;

FIG. 20 is a perspective view of an oxygenator unit constructed in accordance with an embodiment;

FIG. 21 is a front perspective view of a defibrillator unit constructed in accordance with an embodiment of the present invention;

FIG. 22 is a perspective view of the defibrillator unit shown in FIG. 21, illustrating a solar panel and a mobile device for use in conjunction with the defibrillator unit in accordance with an embodiment;

FIG. 23 is a rear perspective view of the defibrillator unit shown in FIG. 21;

FIG. 24 is a view similar to FIG. 23, except that a battery is removed from the defibrillator unit;

FIG. 25 is a perspective view of a resuscitation device constructed in accordance with an embodiment;

FIG. 26 is a perspective view of a resuscitation device constructed in accordance with an embodiment, the resuscitation device including defibrillator and oxygenator units;

FIG. 27 is a perspective view of the defibrillator unit shown in FIG. 26;

FIG. 28 is a perspective view of a resuscitation device constructed in accordance with an embodiment;

FIG. 29 is an exploded perspective view of the resuscitation device shown in FIG. 28;

FIG. 30 is a perspective view of a resuscitation device constructed in accordance with an embodiment, the resuscitation device including defibrillator and oxygenator units;

FIG. 31 is a perspective view of the defibrillator unit shown in FIG. 30;

FIG. 32 is a perspective view of a resuscitation device constructed in accordance with an embodiment, the resuscitation device including defibrillator and oxygenator units;

FIG. 33 is an exploded perspective view of the resuscitation device shown in FIG. 32;

FIG. 34 is a perspective view of a resuscitation device constructed in accordance with an embodiment, the resuscitation device including defibrillator and oxygenator units;

FIG. 35 is an exploded perspective view of the resuscitation device shown in FIG. 34;

FIG. 36 is a perspective view of a resuscitation device constructed in accordance with an embodiment, the resuscitation device including defibrillator and oxygenator units;

FIG. 37 is an exploded perspective view of the resuscitation device shown in FIG. 36;

FIGS. 38 and 39 are schematic views of an adapter constructed in accordance with an embodiment;

FIGS. 40 and 41 are schematic views of an adapter constructed in accordance with an embodiment;

FIG. 42 is a perspective view of the adapter shown in FIGS. 40 and 41; and

FIG. 43 is a view of an adapter constructed in accordance with an embodiment.

### DESCRIPTION OF EXEMPLARY EMBODIMENTS

Embodiments are now discussed in more detail referring to the drawings that accompany the present application. In the accompanying drawings, like and/or corresponding elements are referred to by like reference numbers.

Various embodiments are disclosed herein; however, it is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that covered or claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

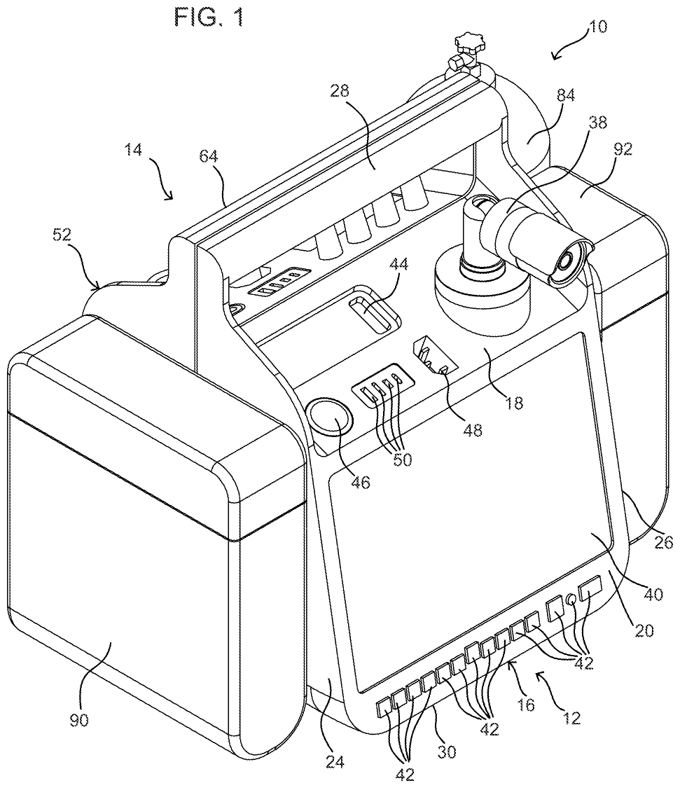

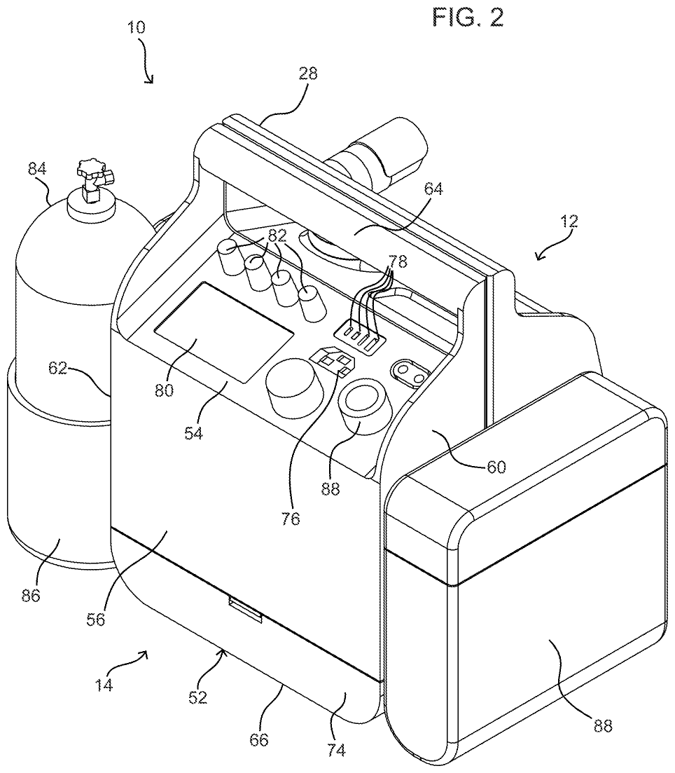

FIGS. 1 and 2 illustrate a resuscitation device 10 constructed in accordance with an embodiment. In one embodiment, the resuscitation device 10 is adapted for use in initiating and/or providing cardiopulmonary resuscitation to patients with emergency medical conditions, such as patients having a cardiac arrest and/or respiratory failure. In one embodiment, the resuscitation device 10 includes an automatic external defibrillator (“AED”) unit 12 and a ventilator unit 14, which can be assembled together so that they can be carried together as a single assembly. In other embodiments, the AED unit 12 and the ventilator unit 14 can be disassembled from one another so that each of them can function as a standalone unit (see FIG. 4). The AED unit 12 and the ventilator unit 14 will be discussed individually hereinbelow.

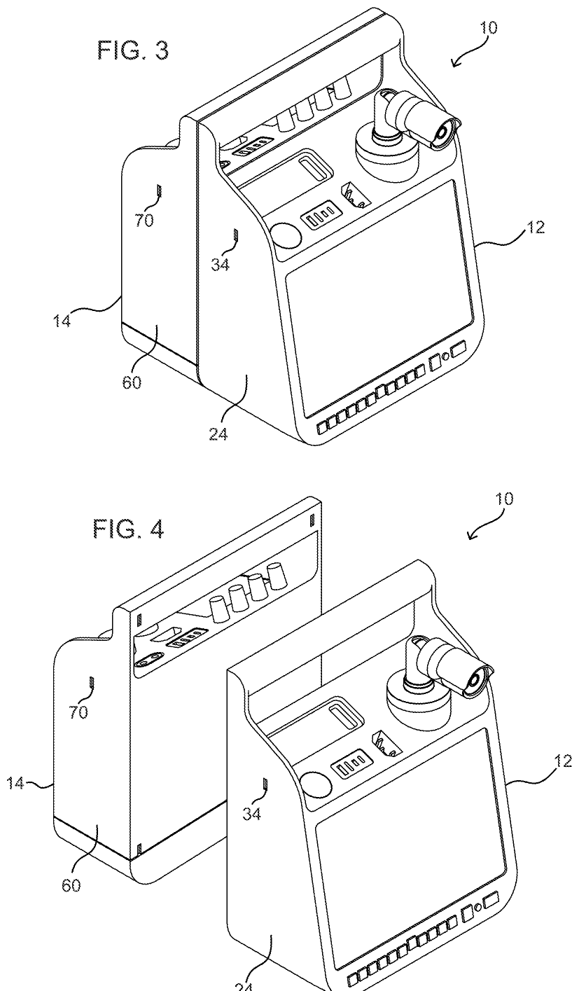

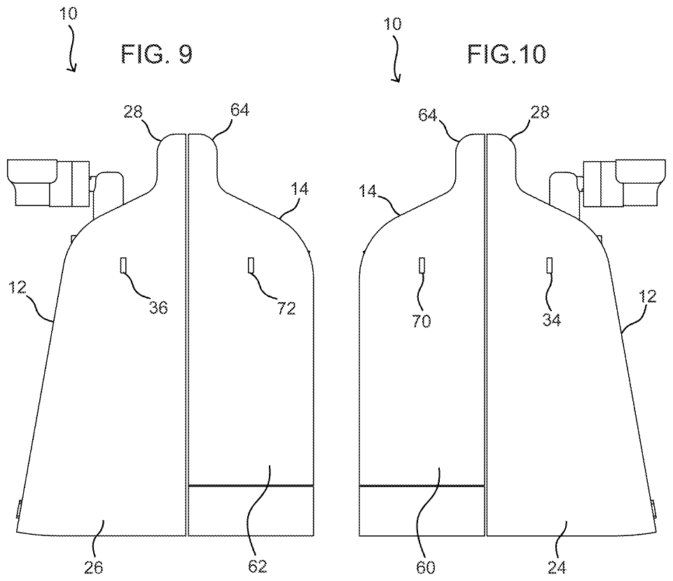

Referring to FIGS. 1 and 2, the AED unit 12 includes a housing 16 (hereinafter “AED housing”) having an upper side 18, front and rear sides 20, 22 (see also FIG. 11) and a pair of lateral sides 24, 26 (see also FIGS. 9 and 10). In one embodiment, the AED housing 16 has a handle 28 extending between the lateral sides 24, 26, and a substantially flat and horizontal bottom 30 (see also FIG. 8) for supporting the AED unit 12 on a supporting surface, such as a floor, a table top, etc. In one embodiment, the AED housing 16 includes one or more slots 32, which are formed in the rear side 22 (see FIG. 11), one or more slots 34, which are formed on the lateral side 24 (only one shown in FIGS. 3, 4 and 10), and one or more slots 36, which are formed on the lateral side 26 (only one shown in FIG. 9), for purposes to be discussed hereinbelow.

In one embodiment, the AED housing 16 is equipped with a wireless video conferencing system for allowing a user of the resuscitation device 10 (e.g., medical personnel, such as an emergency medical responder) to conduct a video conference with another medical personnel (e.g., a medical doctor) in the provision of medical care to a patient. In one embodiment, a video camera 38 is rotatably and/or pivotally mounted on the upper side 18 of the AED housing 16 for taking video images (e.g., video images of the user of the resuscitation device 10 or the patient) for transmission of same to a remote communication device (e.g., a computer, a mobile communication device, etc., located at the location of the other medical personnel). In one embodiment, a video screen 40 is attached to the front side 20 for displaying moving or still visual images (e.g., a video of the other medical personnel, medical and/or telemedicine information, vital signs of the patient detected or monitored by the resuscitation device 10, etc.). In one embodiment, the screen 40 is a touch screen such that the operation of the AED unit 12, including the video conference feature, can be controlled by touching various virtual controls on the screen. In an alternate embodiment, the screen 40 is a non-touch screen, and the operation of the AED unit 12 is controlled via one or more controls 42 (see, e.g., FIG. 1) provided on the AED housing 16. One or more speakers (not shown) and one or more microphones (not shown) are provided in or on the AED housing 16 for allowing wireless audio or audiovisual communication with a remote communication device.

In one embodiment, a wireless communication system (not shown) is provided in the AED unit 12 for allowing wireless communication between the AED unit 12 and one or more remote computerized systems, such remote computer servers, mobile communication devices, stationary computer devices, etc. In one embodiment, the wireless communication system can be any conventional communication device, including, without limitation, a cellular device, a satellite device, a Wi-Fi device and a Bluetooth device.

In one embodiment, the AED unit 12 includes an electrical connector 44 provided on the upper side 18 of the AED housing 16 for electrical connection with defibrillator electrodes or pads, as well as a shock switch 46 for delivering an electrical charge or shock to the defibrillator electrodes. In another embodiment, the AED unit 12 includes an electrical connector 48 for connection to an electrical source for providing electricity to electrical components provided in the AED unit 12, such as an internal battery. In one embodiment, the AED unit 12 is equipped with a plurality of electrical connectors 50 for providing electrical and/or data connection with one or more external devices, such as mobile communication devices. In one embodiment, the electrical connectors 50 include one or more of USB connectors and other conventional electrical and/or data connectors. In one embodiment, the AED unit 12 is configured, and includes electronic and electrical circuitry, so as to be powered by one or more external devices having power sources (e.g., one or mobile phones, tablet computers, laptop computers, portable generators, external batteries, such as battery packs and vehicle batteries, wall outlets, solar panels, etc.) connected to the connectors 50.

In one embodiment, the AED unit 12 is provided with one or more components that are included in conventional AED devices, such as those sold by Cardiac Science under the designations POWERHEART® G3 and POWERHEART® G5, by Defibtech, LLC under the designation Lifeline, and by Philips under the designations HeartStart FR3, HeartStart FRx, HeartStart OnSite and HeartStart Home. For instance, the AED unit 12 includes electrical and/or electronic circuitry and components, including without limitation a capacitor, for selectively delivering an electrical charge to defibrillator electrodes connected thereto and/or for sensing and/or monitoring electrical activity of a patient's heart.

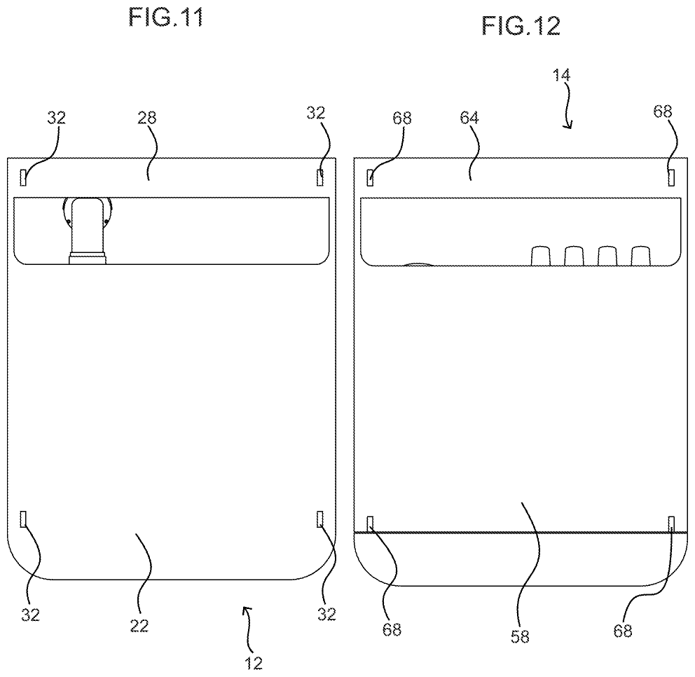

With reference to FIGS. 1 and 2, the ventilator unit 14 includes a housing 52 (hereinafter “ventilator housing”) having an upper side 54, front and rear sides 56, 58 (see also FIG. 12) and a pair of lateral sides 60, 62 (see also FIGS. 9 and 10). In one embodiment, the ventilator housing 52 includes a handle 64 extending between the lateral sides 60, 62. In another embodiment, the ventilator housing 52 includes a substantially flat and horizontal bottom 66 (see also FIG. 8) for supporting the ventilator unit 12 on a supporting surface, such as a floor, a table top, etc. In one embodiment, the ventilator housing 52 includes one or more slots 68, which are formed in the rear side 58 (see FIG. 12), one or more slots 70, which are formed on the lateral side 60 (only one shown in FIGS. 3, 4 and 10), and one or more slots 72, which are formed on the lateral side 62 (only one shown in FIG. 9), for purposes to be discussed hereinbelow. The ventilator housing 52 also includes an access panel 74 (see FIGS. 2 and 6) for allowing access to internal components of the ventilator unit 14 (e.g., an internal rechargeable battery).

In one embodiment, the ventilator unit 14 includes an electrical connector 76 provided on the upper side 54 of the ventilator housing 52 for connection to an electrical source (e.g., a wall outlet or a solar panel) for providing electricity to electrical components provided in the ventilator unit 14, such as a rechargeable battery. In another embodiment, the ventilator unit 14 is equipped with a plurality of electrical connectors 78 for providing electrical and/or data connection with one or more external devices, such as mobile communication devices. In one embodiment, the ventilator unit 14 is configured, and include electrical and/or electronic circuitry, so as to be powered by one or more external devices (e.g., one or mobile phones, tablet computers, laptop computers, portable generators, external batteries, such as battery packs and vehicle batteries, wall outlets, solar panels, etc.) connected to the connectors 78. In other embodiments, the ventilator unit 14 is equipped with a display panel 80 and a plurality of controls 82 for controlling the operation of the ventilator unit 14.

In one embodiment, the ventilator unit 14 includes an oxygen supply 84 (e.g., an oxygen tank) carried in a holder 86 which is removably attached to the lateral side 62 of the ventilator unit 14. The ventilator unit 14 is also equipped with one or more ports 88 (only one shown in FIG. 2) for connection to the oxygen supply 84 and to one or more air hoses (not shown).

In one embodiment, the ventilator unit 14 is provided with one or more components that are included in conventional ventilator devices, such as those sold by Allied Healthcare under the designations EPV100, EPV200, MCV200, MCV100, AutoVent 2000, AutoVent 3000 and AutoVent 4000, by AutoMedX under the designation Save II Ventilator, and by Philips under the designations Trilogy Evo, Trilogy200, Trilogy100. For instance, the ventilator unit 14 includes mechanical components and/or electrical and/or electronic circuitry and components for performing various functions of the ventilator unit 14.

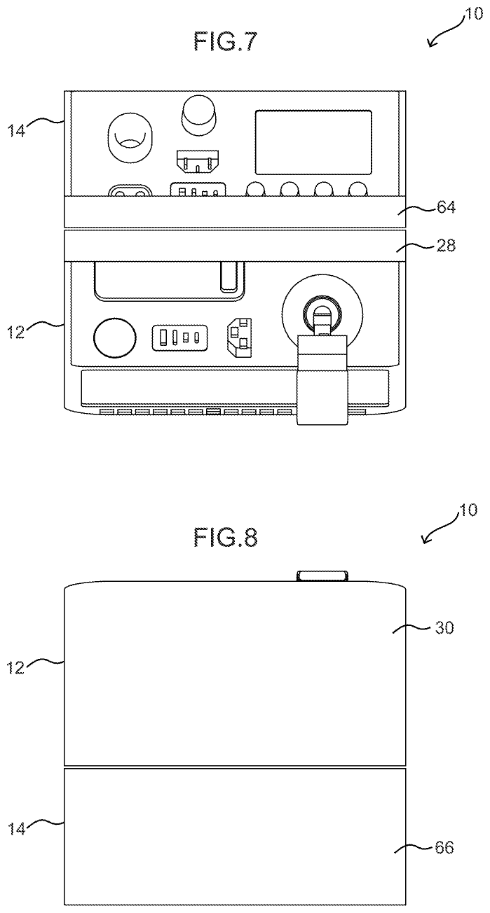

Referring back to FIGS. 1 and 2, the AED unit 12 and the ventilator unit 14 are configured so that they can be combined together to form a single portable assembly (see also FIGS. 3, 7, 8, 9 and 10). In one embodiment, each of the AED unit 12 and the ventilator unit 14 is provided with a releasable mechanism for removably attaching and/or locking the AED and ventilator units 12, 14 to each other. In one embodiment, the attachment mechanism includes one or more of hook and notch mechanisms, hook and loop mechanism (such as the devices sold under the designation VELCRO), tongue and groove mechanisms and magnetic mechanisms. For instance, hooks, pegs or ribs can be removably or fixedly attached to the slots 32 of the AED unit 12 or the slots 68 of the ventilator unit 14 for releasably mating with the slots of the other one of the AED unit 12 or the ventilator unit 14. Magnets can also be removably or fixedly placed in the slots 32 of the AED unit 12 and the slots 68 of the ventilator unit 14 for magnetically attaching the AED unit 12 to the ventilator unit 14. In another embodiment, each of the AED unit 12 and the ventilator unit 14 is adapted to be used as a standalone device upon disassembly from each other.

In one embodiment, the size and configuration of the rear side 22 of the AED unit 12 substantially match with (i.e., are substantially identical to) those of the rear side 58 of the ventilator unit 14 (see FIGS. 11 and 12) such that the AED unit 12 and the ventilator unit 14 can be assembled with one another in an abutting fashion (see FIGS. 9 and 10). In another embodiment, when the AED unit 12 and the ventilator unit 14 are combined together, the handle 28 of the AED unit 12 abuts against the handle 64 of the ventilator unit 14 such that the handles 28, 64 form a single combined handle that can be gripped by a user for carrying the combined assembly. In other embodiments, the bottom 30 of the AED unit 12 and the bottom 66 of the ventilator unit 14 form a combined, substantially continuous bottom (see FIG. 8) for supporting the combined assembly on a supporting surface.

Referring back to FIGS. 1 and 2, the resuscitation device 10 can be provided with one or more pouches 88, 90 for carrying various accessories for the AED unit 12 and/or the defibrillator unit 14, such as defibrillator pads or electrodes, hoses and masks for the ventilator unit 14 and an adapter (to be discussed below). In one embodiment, the pouches 88, 90 can be removably connected to the AED unit 12 and/or the defibrillator unit 14 via any conventional mechanisms, such as hook and notch mechanisms, magnetic mechanisms, hook and loop mechanism (such as the devices sold under the designation VELCRO), etc. For instance, one or more hooks, pegs or ribs can be removably or fixedly attached to the slots 34, 36 of the AED unit 12 and/or the slots 70, 72 of the ventilator unit 14 for fastening the holder 86, the pouch 88 and/or the pouch 90 to the AED unit 12 and/or the ventilator unit 14. One or more magnets can also be removably or fixedly placed in the slots 34, 36 of the AED unit 12 and the slots 70, 72 of the ventilator unit 14 for magnetically attaching the holder 86, the pouch 88 and/or the pouch 90 to the AED unit 12 and/or the ventilator unit 14. In another embodiment, at least one of the pouches 88, 90 are attached to the AED unit 12 and the ventilator unit 14 so as to secure the AED and defibrillator units 12, 14 to one another, thereby inhibiting them from being disassembled from each other until the attached pouch or pouches 88, 90 are removed from the AED and defibrillator units 12, 14.

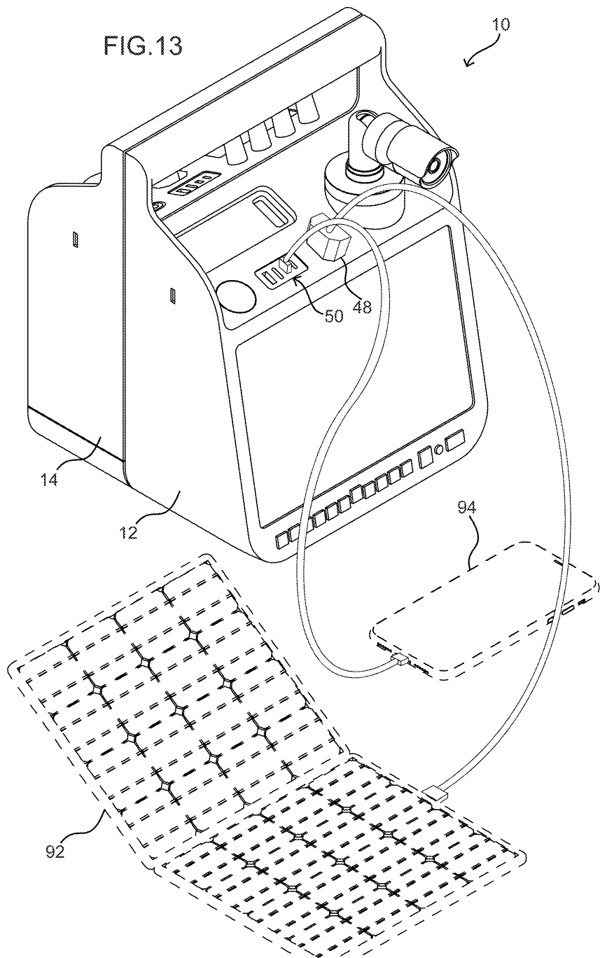

With reference to FIG. 13, each of the electrical connectors 48, 76 of the AED unit 12 and the ventilator unit 14, respectively, is adapted to be connected to an electrical source, such as an AC electric source (e.g., a wall outlet), a DC electrical source or a solar panel 92, for charging a corresponding one of the batteries provided in the AED and ventilator units 14 and 16. In other embodiments, the AED unit 12 and/or the ventilator unit 14 can be connected to external devices, such as mobile communication devices 94, via one or more of the electrical connectors 50, 78, respectively for powering a corresponding one of the AED and ventilator units 14 , 16.

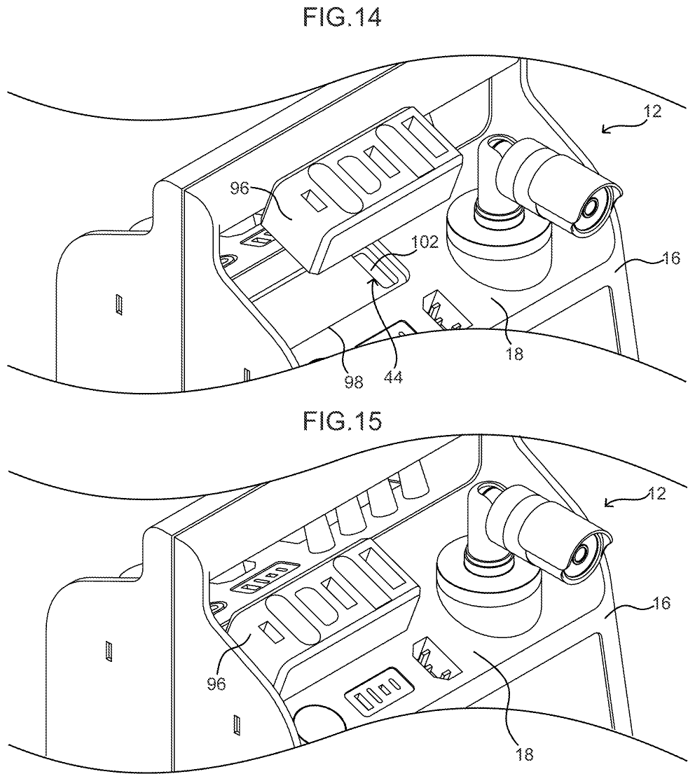

Now referring to FIGS. 14 and 15, an adapter 96 is provided for removable connection with the AED unit 12 in accordance with one embodiment. In one embodiment, the adapter 96 is configured to be received in a receiving bay 98 provided in the upper side 18 of the AED housing 16 and surrounding the electrical connector 44. In another embodiment, the adapter 96 includes a plug 100, which is provided on one side of the adapter 96 so as to be plugged into a receptacle 102 of the electrical connector 44, and a plurality of electrical connectors 104, each of which is configured for electrical connection with a specific set of defibrillator electrodes having different connectors. In this manner, the AED unit 12 can be used in conjunction with a number of different defibrillator electrodes (e.g., defibrillator electrodes manufactured by different manufacturers with different specifications and having distinct electrical connectors).

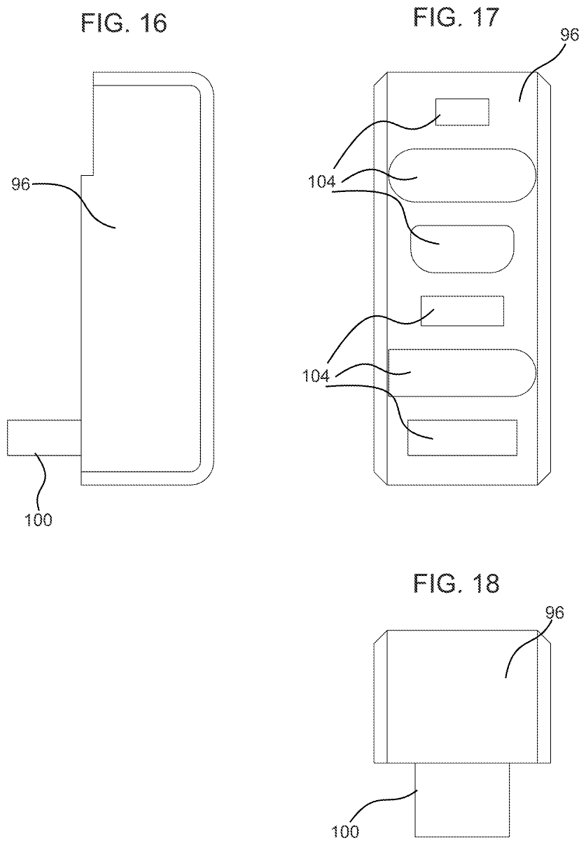

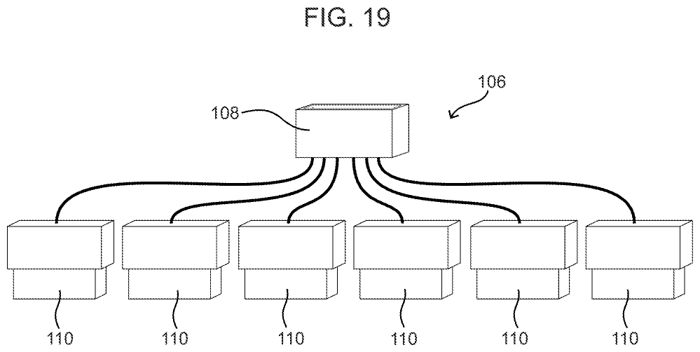

FIG. 19 illustrates an adapter 106 constructed in accordance with an alternate embodiment. The adapter 106 has a connector 108 for connection with a connector of a specific set of defibrillator electrodes (e.g., electrodes manufactured by one specific manufacturer with its own specification). The adapter 106 also includes a plurality of different connectors 110, each of which is adapted to be connected to a connector of a specific AED device. In this manner, the specific set of defibrillator electrodes connected to the adapter 106 can be used in conjunction with a number of different AED devices. The plug 100 of the adapter 96 illustrated in FIGS. 14-18 can be coupled to the connector 108 of the adapter 106 of FIG. 19 such that many different defibrillator electrodes can be used in conjunction with many different AED devices.

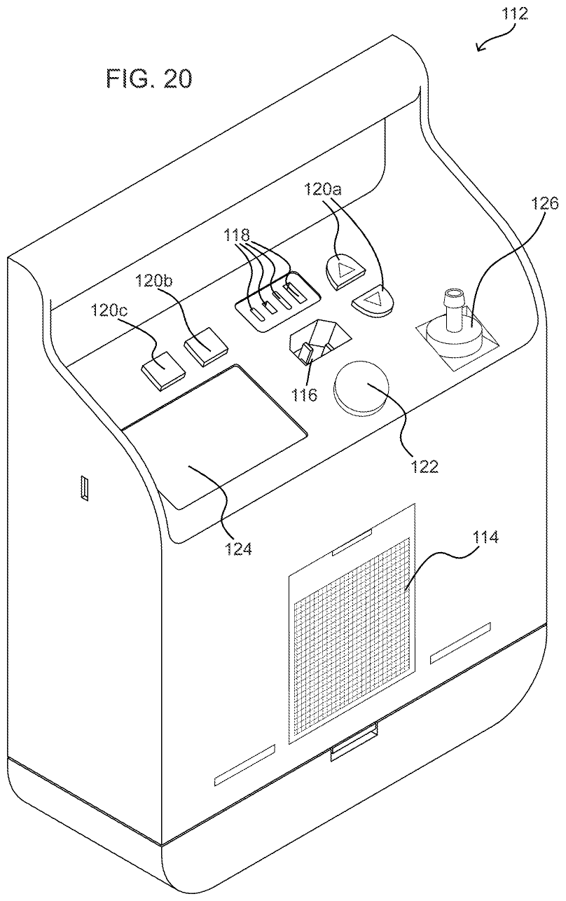

Now referring to FIG. 20, there is shown an oxygenator unit 112 constructed in accordance with one embodiment. In one embodiment, the oxygenator unit 112 can be used as a standalone unit. In another embodiment, the oxygenator unit 112 can be used in conjunction with the AED unit 12 shown in FIGS. 1 and 2 by assembling the oxygenator unit 112 with the AED unit 12 in a manner substantially identical or similar to the manner in which the ventilator unit 14 is assembled with the AED unit 12. In this manner, the AED unit 12 can be equipped with the ventilator unit 14 or the oxygenator unit 112 to provide different functionalities to meet different requirements and/or preferences. In one embodiment, the oxygenator unit 112 includes a removable particle filter 114 for filtering air going in and out of the oxygenator unit 112. In a further embodiment, the oxygenator unit 112 includes a battery (either fixed or removable) for supplying electricity to electrical components housed in the oxygenator unit 112. In another embodiment, the oxygenator unit 112 is equipped with an electrical connector 116 for connection with an electrical source, such as an AC power source (e.g., a wall outlet) and a solar panel, for charging the battery 114 and/or for providing electricity to the various electrical components of the oxygenator unit 112. In other embodiments, the oxygenator unit 112 has a plurality of connectors 118 (e.g., USB connectors) for connection to external electronic devices (e.g., one or mobile phones, tablet computers, laptop computers, portable generators, external batteries, such as battery packs and vehicle batteries, wall outlets, solar panels, etc.), which can provide electrical power to the oxygenator unit 112. In a further embodiment, the oxygenator unit 112 includes flow rate controls 120a, a volume on/off control 120b, no-breath warning alarm on/off control 120c, a power control button 122, a screen 124, and a connector 126 for attachment to a nasal cannula.

In one embodiment, the oxygenator unit 112 is provided with one or more components that are included in conventional oxygenator devices, such as those sold by Inogen under the designations G3, G4 and G5, by Philips under the designations SimplyGo and SimplyGo Mini, and by Precision Medical under the designations EasyPulse and Live Active Five. For instance, the oxygenator unit 112 includes mechanical components and/or electrical and/or electronic circuitry and components for performing various functions of the oxygenator unit 112.

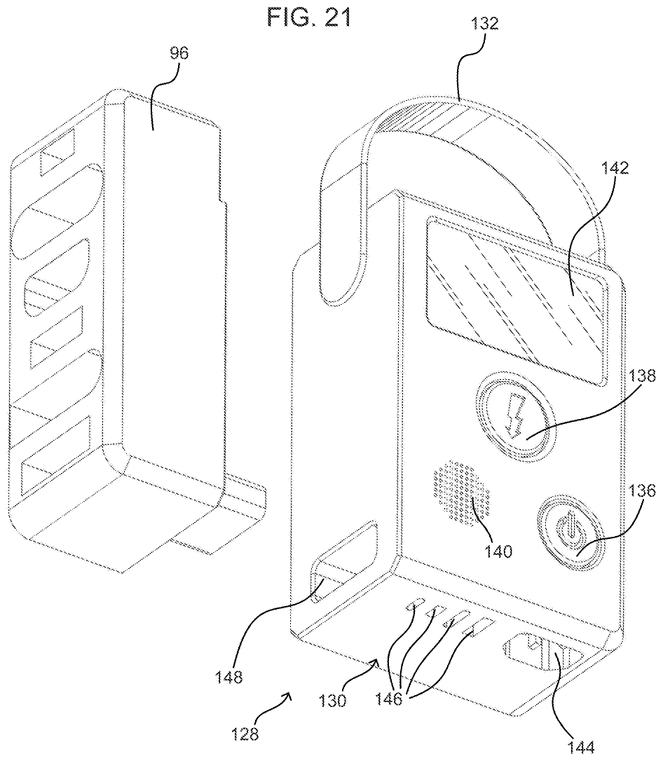

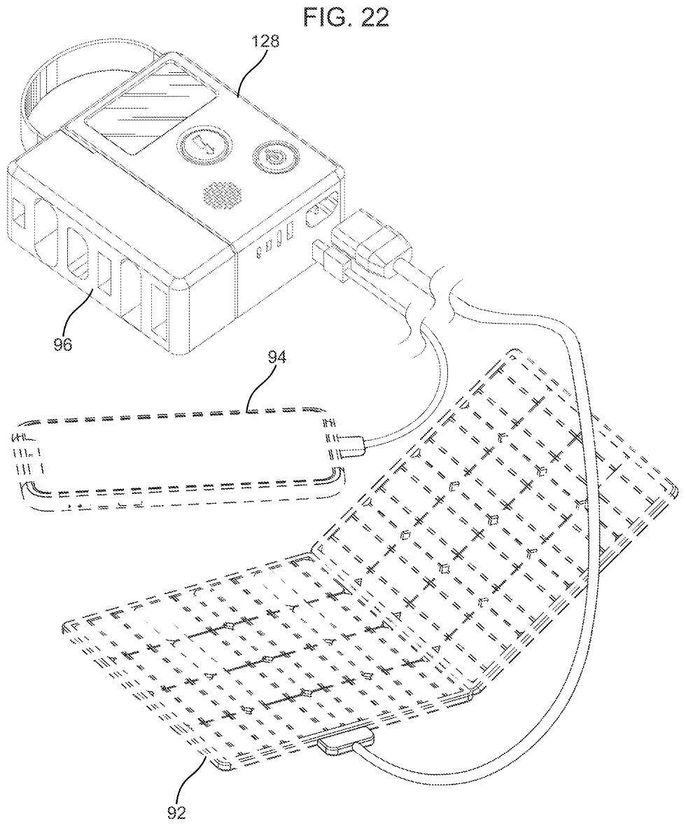

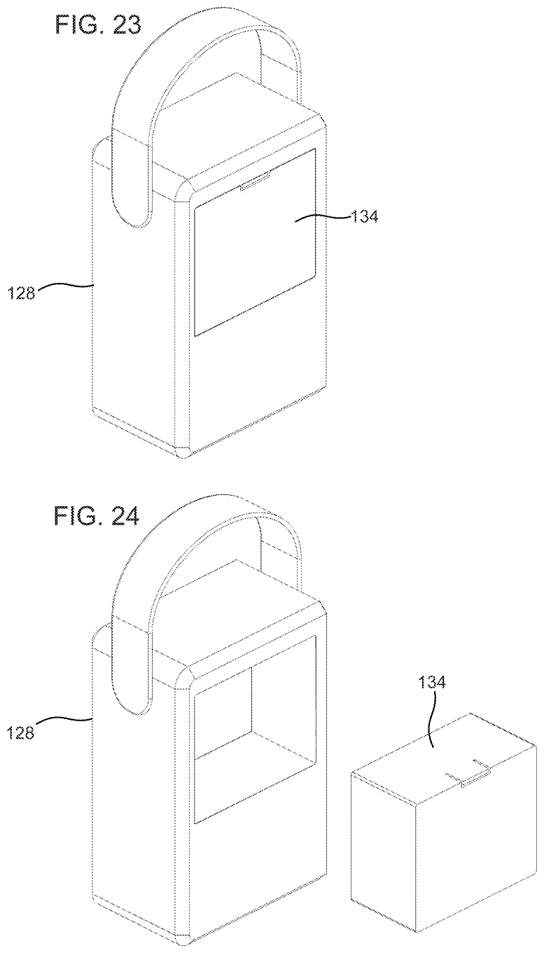

FIG. 21 shows a portable AED unit 128 constructed in accordance with an embodiment. In one embodiment, the ADE unit 128 is small and can be taken to various places, such home or a vehicle. In one embodiment, the AED unit 128 includes a housing 130, a handle 132 attached to the housing 130 and a battery 134 (see FIGS. 23 and 24) removably attached to the housing 130. In other embodiments, the AED unit 128 includes a power button 136, a shock button 138, a speaker 140 for providing audio commands, and a screen 142. In another embodiment, the AED unit 128 includes an electrical connector 144 for connection with an electrical source, such as an AC power source (e.g., a wall outlet) and a solar panel 192 (see, e.g., FIG. 22), for charging the battery 134 and/or for providing electricity to various electrical components of the AED unit 128. In other embodiments, the AED unit 128 has a plurality of connectors 146 (e.g., USB connectors) for connection to external electronic devices, such as mobile phones 94 (see, e.g., FIG. 22), for powering the AED unit 128 from electrical sources (e.g., batteries) of the external electronic devices 94. In a further embodiment, the AED unit 128 includes an electrical connector 148 for connection with the adapter 96 discussed above in connection with FIGS. 14-18.

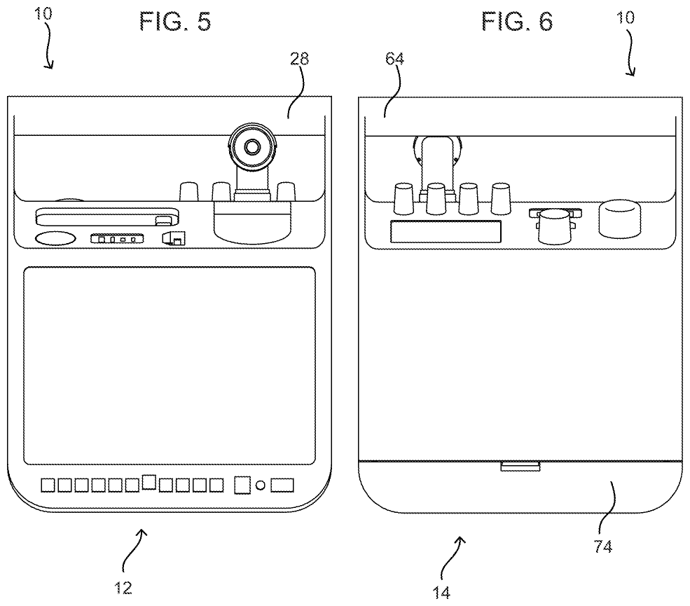

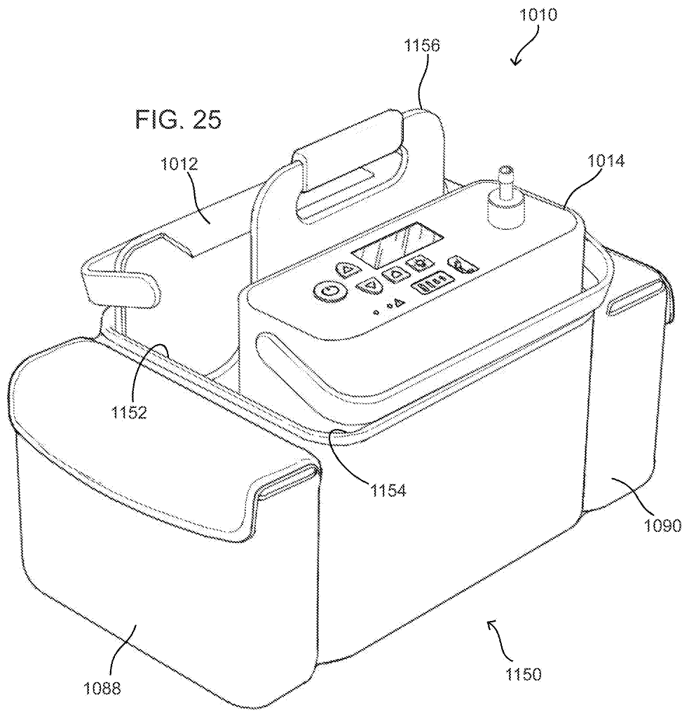

FIG. 25 illustrates a resuscitation device 1010 constructed in accordance with one embodiment. More particularly, the resuscitation device 1010 includes resuscitation units 1012, 1014, which can be assembled together as a single assembly. In one embodiment, the resuscitation unit 1012 includes an AED unit having structural, electrical and/or functional features substantially identical or similar to those discussed above in connection with the AED unit of FIG. 1-15. In another embodiment, the resuscitation unit 1014 includes an oxygenator having structural, electrical and/or functional features substantially identical or similar to those discussed above in connection with the oxygenator shown in FIG. 20. In a further embodiment, the resuscitation unit 1014 may include a ventilator having structural, electrical and/or functional features substantially identical or similar to those discussed above in connection with the ventilator shown in FIGS. 1-15.

In one embodiment, the resuscitation device 1010 includes a basket 1150 for carrying the resuscitation units 1012, 1014 as an assembly. More particularly, the basket 1150 includes a pair of compartments 1152, 1154, each of which is adapted to removably receive a corresponding one of the resuscitation units 1012, 1014. In one embodiment, the basket 1150 includes a handle 1156 projecting between the compartments 1152, 1154 and hence between the resuscitation units 1012, 1014. The basket 1150 also includes a pair of pouches 1088, 1090, each of which is fixedly or removably attached to a corresponding side of the basket 1150.

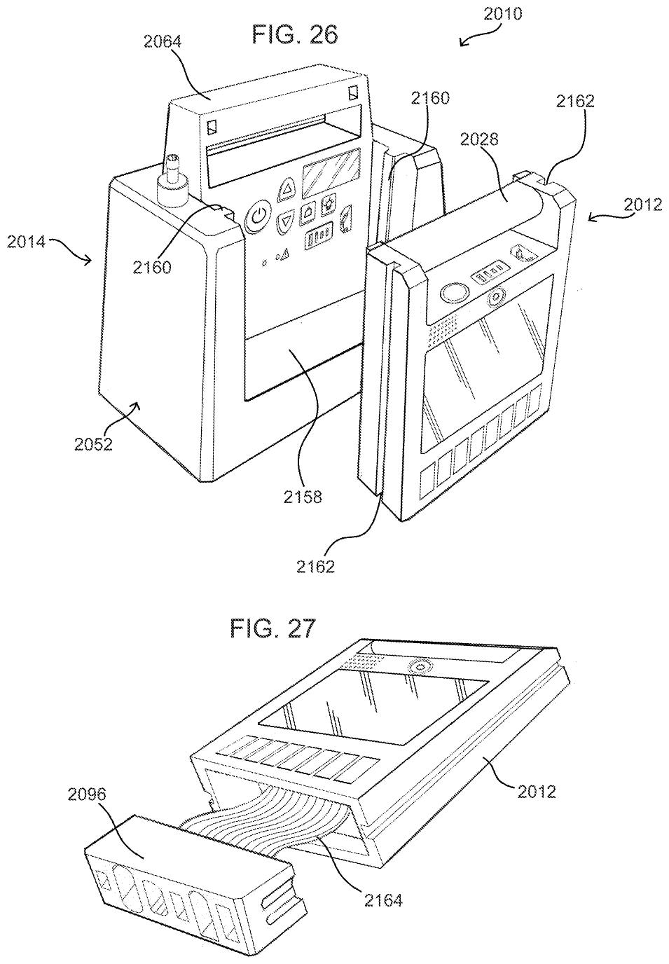

FIG. 26 illustrates a resuscitation device 2010 constructed in accordance with one embodiment. More particularly, the resuscitation device 2010 includes resuscitation units 2012, 2014, which can be assembled together as a single assembly. In one embodiment, the resuscitation unit 2014 includes an oxygenator having structural, electrical and/or functional features substantially identical or similar to those discussed above in connection with the oxygenator 112 shown in FIG. 20. In another embodiment, the resuscitation unit 2014 may include a ventilator having structural, electrical and/or functional features substantially identical or similar to those discussed above in connection with the ventilator shown in FIGS. 1-15. In one embodiment, the resuscitation unit 2014 includes a housing 2052 having a handle 2064 for carrying the resuscitation unit 2014. In another embodiment, the housing 2052 includes a bay 2158 and a pair of tracks 2160 for purposes to be discussed below.

Still referring to FIG. 26, the resuscitation unit 2012 includes an AED unit having structural, electrical and/or functional features substantially identical or similar to those discussed above in connection with the AED unit 12 illustrated in FIGS. 1-15. The resuscitation unit 2012 includes a pair of grooves 2162, each of which is adapted to mate with a corresponding one of the tracks 2160 of the resuscitation unit 2014. In one embodiment, the resuscitation unit 2012 is inserted into the bay 2158 of the housing 2052 of the resuscitation unit 2014. In one embodiment, the tracks 2160 of the resuscitation unit 2014 and the grooves 2162 of the resuscitation unit 2012 are adapted to releasably lock the resuscitation units 2012, 2014 to each other. In another embodiment, additional or alternate mechanisms (e.g., a latch mechanism) can be provided for releasably locking the resuscitation units 2012, 2014 to one another. In one embodiment, the resuscitation unit 2012 includes a handle 2028, which can be used for carrying the resuscitation unit 2012 when it the resuscitation unit 2012 is used as a standalone unit. In another embodiment, when the resuscitation units, 2012, 2014 are assembled together, the handle 2028 of the resuscitation unit 2012 abuts the handle 2064 of the resuscitation unit 2014 so as to form a combined single handle, which can be gripped by a user for carrying the combined assembly of the resuscitation units 2012, 2014.

Referring now to FIG. 27, the resuscitation unit 2012 includes an adapter 2096 removable stored in a compartment at a lower end of the AED unit 2012 in accordance with one embodiment. More particularly, the adapter 2096 has structural, electrical and/or functional features substantially identical or similar to those of the adapter 96 illustrated in FIGS. 14-18, except that the adapter 2096 is permanently or removably connected to the resuscitation unit 1012 via a wire connector 2164.

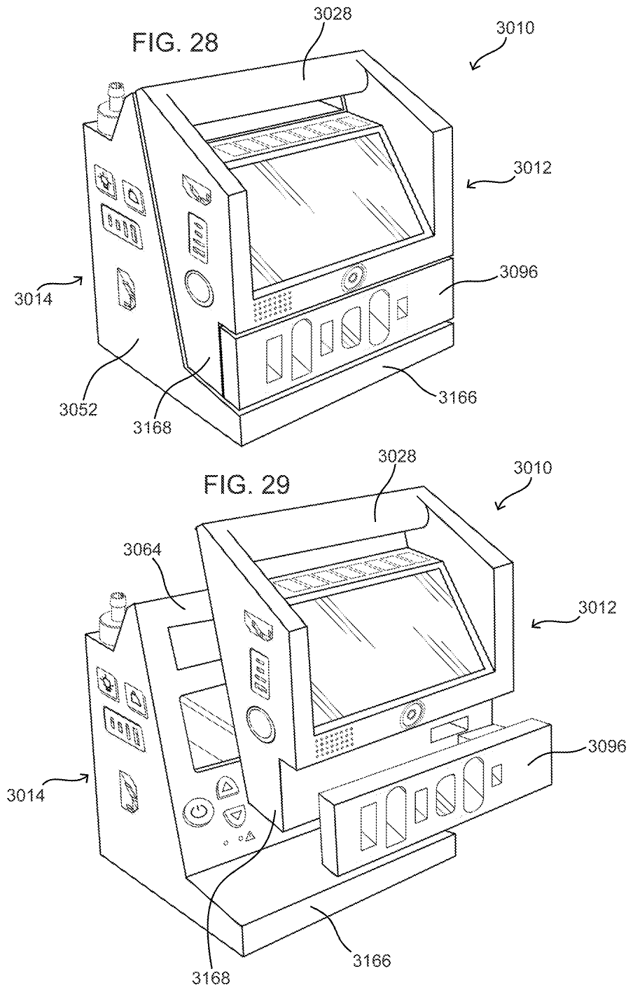

FIGS. 28 and 29 illustrate a resuscitation device 3010 constructed in accordance with one embodiment. More particularly, the resuscitation device 3010 includes resuscitation units 3012, 3014, which can be assembled together as a single assembly. In one embodiment, the resuscitation unit 3014 includes an oxygenator having structural, electrical and/or functional features substantially identical or similar to those discussed above in connection with the oxygenator shown in FIG. 20. In another embodiment, the resuscitation unit 3014 includes a ventilator having structural, electrical and/or functional features substantially identical or similar to those discussed above in connection with the ventilator shown in FIGS. 1-15. In one embodiment, the resuscitation unit 3014 includes a housing 3052 having a handle 3064 for carrying the resuscitation unit 3014 when it is used as a standalone unit. In another embodiment, the housing 3052 includes a laterally projecting ledge 3166 for purposes to be discussed below.

Still referring to FIGS. 28 and 29, the resuscitation unit 3012 includes an AED unit having structural, electrical and/or functional features substantially identical or similar to those discussed above in connection with the AED unit 12 illustrated in FIGS. 1-15. In one embodiment, the resuscitation unit 2012 includes a bottom 3168 that is adapted to be supported on the ledge 3166 of the resuscitation unit 3014 when the resuscitation unit 3012 is assembled with the resuscitation unit 3014. In another embodiment, a locking mechanism (such as a latch mechanism) is provided for releasably locking the resuscitation unit 3012 to the resuscitation unit 3014. In one embodiment, the resuscitation unit 3012 includes a handle 3028, which can be used for carrying the resuscitation unit 3012 when the resuscitation unit 3012 is used as a standalone unit. In another embodiment, when the resuscitation units, 3012, 3014 are assembled together, the handle 3028 of the resuscitation unit 3012 abuts the handle 3064 of the resuscitation unit 3014 so as to form a combined single handle, which can be gripped by a user for carrying the combined assembly of the resuscitation units 3012, 3014.

With continued reference to FIGS. 28 and 29, the resuscitation unit 3012 includes an adapter 3096 removable attached to a lower end of the resuscitation unit 3012 in accordance with one embodiment. More particularly, the adapter 3096 has structural, electrical and/or functional features substantially identical or similar to those of the adapter 96 illustrated in FIGS. 14-18.

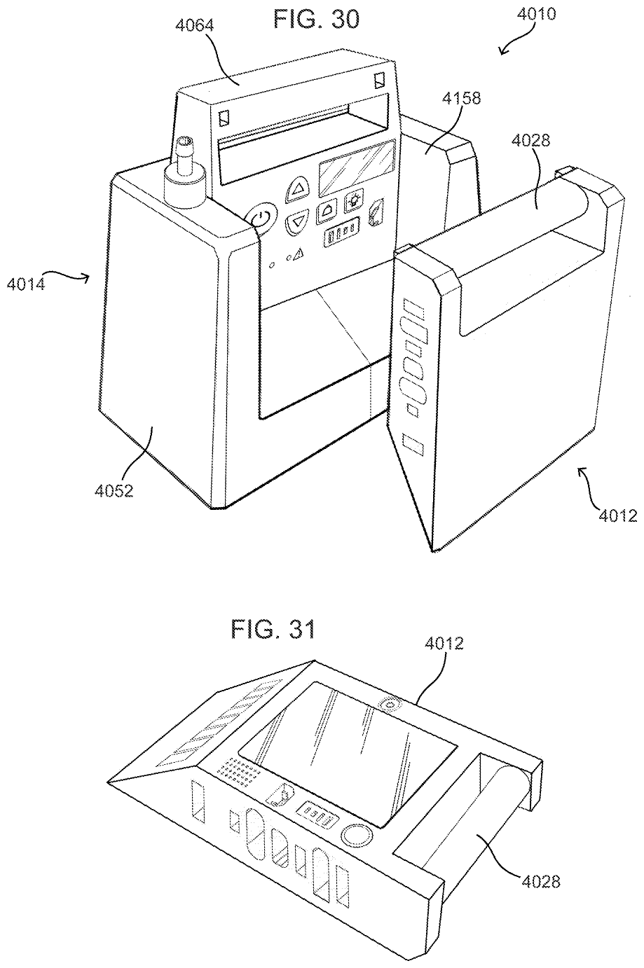

FIGS. 30 and 31 illustrate a resuscitation device 4010 constructed in accordance with one embodiment. More particularly, the resuscitation device 4010 includes resuscitation units 4012, 4014, which can be assembled together as a single assembly. In one embodiment, the resuscitation unit 4014 includes an oxygenator having structural, electrical and/or functional features substantially identical or similar to those discussed above in connection with the oxygenator shown in FIG. 20. In another embodiment, the resuscitation unit 4014 may include a ventilator having structural, electrical and/or functional features substantially identical or similar to those discussed above in connection with the ventilator shown in FIGS. 1-15. In one embodiment, the resuscitation unit 4014 includes a housing 4052 having a handle 4064 for carrying the resuscitation unit 4014 when it is used as a standalone unit. In another embodiment, the housing 4052 includes a bay 4158 for purposes to be discussed below.

Still referring to FIGS. 30 and 31, the resuscitation unit 4012 includes an AED unit having structural, electrical and/or functional features substantially identical or similar to those discussed above in connection with the AED unit 12 illustrated in FIGS. 1-15. In one embodiment, the resuscitation unit 4012 is removably received in the bay 4158 of the resuscitation unit 4014 when the resuscitation unit 4012 is assembled with the resuscitation unit 4014. In another embodiment, a locking mechanism (such as a latch mechanism) is provided for releasably locking the resuscitation unit 4012 to the resuscitation unit 4014. In one embodiment, the resuscitation unit 4012 includes a handle 4028 which can be used for carrying the resuscitation unit 4012 when it is used as a standalone unit. In another embodiment, when the resuscitation units, 4012, 4014 are assembled together, the handle 4028 of the resuscitation unit 4012 abuts the handle 4064 of the resuscitation unit 4014 so as to form a combined single handle, which can be gripped by a user for carrying the combined assembly of the resuscitation units 4012, 4014. Unlike the AED unit 12 illustrated in FIGS. 1-15, the adapter 96 shown in FIGS. 14-18 is built directly and integrally into a side of the resuscitation unit 4012 in accordance with one embodiment.

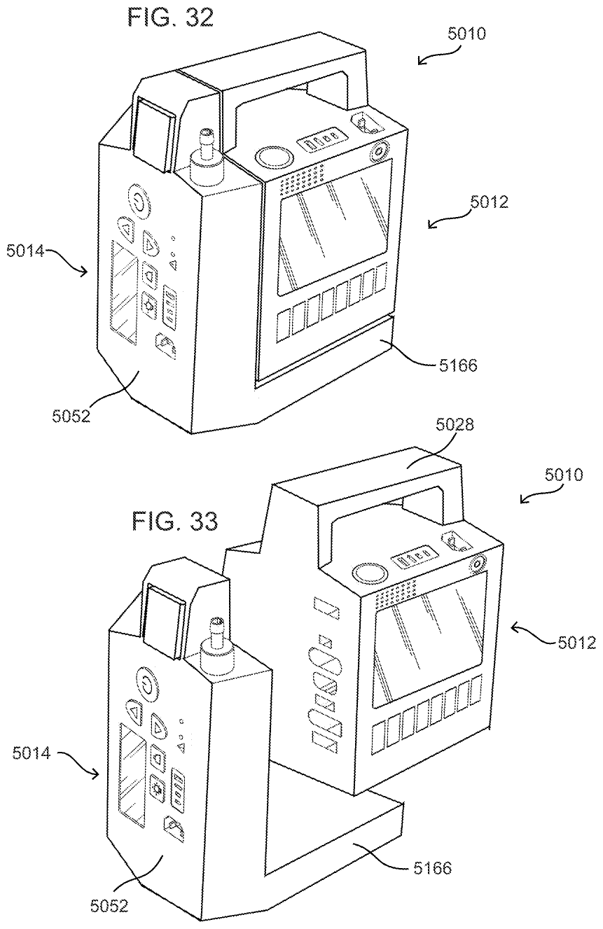

FIGS. 32 and 33 illustrate a resuscitation device 5010 constructed in accordance with one embodiment. More particularly, the resuscitation device 5010 includes resuscitation units 5012, 5014, which can be assembled together as a single assembly. In one embodiment, the resuscitation unit 5014 includes an oxygenator having structural, electrical and/or functional features substantially identical or similar to those discussed above in connection with the oxygenator shown in FIG. 20. In another embodiment, the resuscitation unit 5014 may include a ventilator having structural, electrical and/or functional features substantially identical or similar to those discussed above in connection with the ventilator shown in FIGS. 1-15. In one embodiment, the resuscitation unit 5014 includes a housing 5052 having a ledge 5166 for purposes to be discussed below.

Still referring to FIGS. 32 and 33, in one embodiment, the resuscitation unit 5012 includes an AED unit having structural, electrical and/or functional features substantially identical or similar to those discussed above in connection with the AED unit 12 illustrated in FIGS. 1-15. In one embodiment, the resuscitation unit 5012 is removably received on the ledge 5166 of the resuscitation unit 5014 when the resuscitation unit 5012 is assembled with the resuscitation unit 5014. In another embodiment, a locking mechanism (such as a latch mechanism) is provided for releasably locking the resuscitation units 5012, 5014 with one another. In one embodiment, the resuscitation unit 5012 includes a handle 5028 which can be used for carrying the resuscitation unit 5012 when it is used as a standalone unit. In another embodiment, when the resuscitation unit 5012 is assembled and locked with the resuscitation unit 5014, the handle 5028 of the resuscitation unit 5012 abuts the handle 5064 of the resuscitation unit 5014 so as to form a combined single handle, which can be gripped by a user for carrying the combined assembly of the resuscitation units 5012, 5014. Unlike the AED unit 12 illustrated in FIGS. 1-15, the adapter 96 shown in FIGS. 14-18 is built directly and integrally into a side of the resuscitation unit 5012 in accordance with one embodiment.

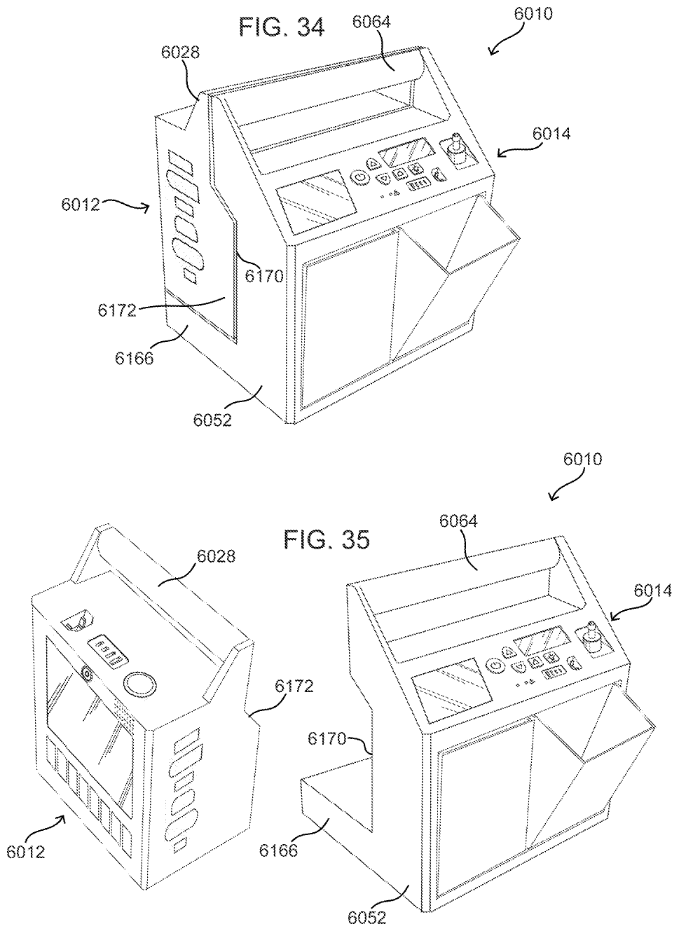

FIGS. 34 and 35 illustrate a resuscitation device 6010 constructed in accordance with one embodiment. More particularly, the resuscitation device 6010 includes resuscitation units 6012, 014, which can be assembled together as a single assembly. In one embodiment, the resuscitation unit 6014 includes an oxygenator having structural, electrical and/or functional features substantially identical or similar to those discussed above in connection with the oxygenator shown in FIG. 20. In another embodiment, the resuscitation unit 6014 includes a ventilator having structural, electrical and/or functional features substantially identical or similar to those discussed above in connection with the ventilator shown in FIGS. 1-15. In one embodiment, the resuscitation unit 6014 includes a housing 6052 having a handle 6064 for carrying the resuscitation unit 6014 when it is used as a standalone unit. In another embodiment, the housing 6052 includes a ledge 6166 and a slot 6170 for purposes to be discussed below.

Still referring to FIGS. 34 and 35, in one embodiment, the resuscitation unit 6012 includes an AED unit having structural, electrical and/or functional features substantially identical or similar to those discussed above in connection with the AED unit 12 illustrated in FIGS. 1-15. In one embodiment, the resuscitation unit is removably received over a ledge 6166 of the resuscitation unit 6014 when the resuscitation unit 6012 is assembled with the resuscitation unit 6014. The resuscitation unit 6012 includes a side projection 6172 which is adapted to be received in the slot 6170 of the resuscitation unit 6014. In another embodiment, a locking mechanism (such as a latch mechanism) is provided for releasably locking the resuscitation units 6012, 6014 with one another. In one embodiment, the resuscitation unit 6012 includes a handle 6028, which can be used for carrying the resuscitation unit 6012 when it is used as a standalone unit. In another embodiment, when the resuscitation units, 6012, 6014 are assembled together, the handle 6028 of the resuscitation unit 6012 abuts the handle 6064 of the resuscitation unit 6014 so as to form a combined single handle, which can be gripped by a user for carrying the combined assembly of the resuscitation units 6012, 6014. Unlike the AED unit 12 illustrated in FIGS. 1-15, the adapter 96 shown in FIGS. 14-18 is built directly and integrally into a side of the resuscitation unit 6012 in accordance with one embodiment.

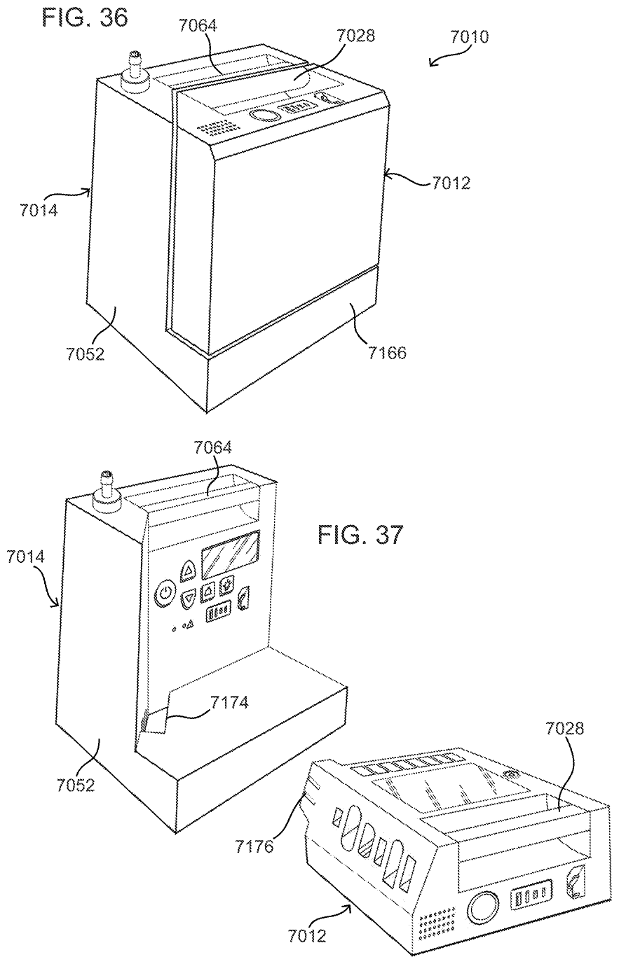

FIGS. 36 and 37 illustrate a resuscitation device 7010 constructed in accordance with one embodiment. In one embodiment, the resuscitation device 7010 includes resuscitation units 7012, 7014, which can be assembled together to form a single assembly. In one embodiment, the resuscitation unit 7014 includes an oxygenator having structural, electrical and/or functional features substantially identical or similar to those discussed above in connection with the oxygenator shown in FIG. 20. In another embodiment, the resuscitation unit 7014 may include a ventilator having structural, electrical and/or functional features substantially identical or similar to those discussed above in connection with the ventilator 14 shown in FIGS. 1-15. In one embodiment, the resuscitation unit 7014 includes a housing 7052 having a handle 7064 for carrying the resuscitation unit 7014 when it is used as a standalone unit. In another embodiment, the housing 7052 includes a ledge 7166 and a recess 7174 for purposes to be discussed below.

Still referring to FIGS. 36 and 37, in one embodiment, the resuscitation unit 7012 includes an AED unit having structural, electrical and/or functional features substantially identical or similar to those discussed above in connection with the AED unit 12 illustrated in FIGS. 1-15. In one embodiment, the resuscitation unit 7012 is removably received on the ledge 7166 of the resuscitation unit 7014 when the resuscitation unit 7012 is assembled with the resuscitation unit 7014. The resuscitation unit 7012 includes a bottom projection 7176, which is adapted to be received in the recess 7174 of the resuscitation unit 7014. In another embodiment, a locking mechanism (such as a latch mechanism) is provided for releasably locking the resuscitation units 7012, 7014 with one another. In one embodiment, the resuscitation unit 7014 includes a handle 7028, which can be used for carrying the resuscitation unit 7014 when it is used as a standalone unit. In another embodiment, when the resuscitation units, 7012, 7014 are assembled together, the handle 7028 of the resuscitation unit 7012 abuts the handle 7064 of the resuscitation unit 7014 so as to form a combined single handle, which can be gripped by a user for carrying the combined assembly of the resuscitation units 7012, 7014. Unlike the AED unit 12 illustrated in FIGS. 1-15, the adapter 96 shown in FIGS. 14-18 is built directly and integrally into a side of the resuscitation unit 7012 in accordance with one embodiment.

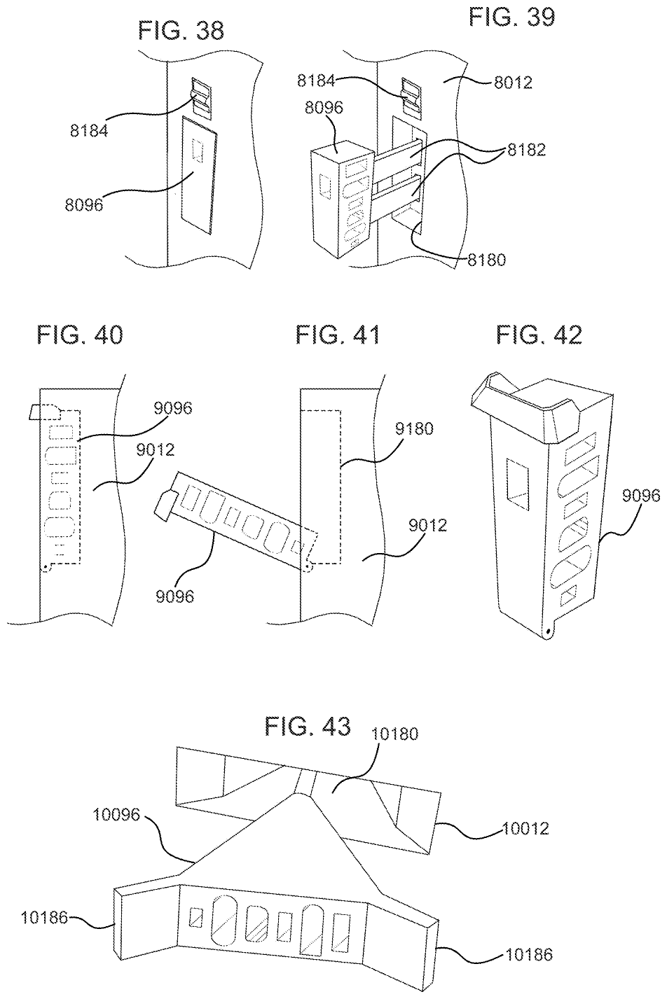

FIGS. 38 and 39 illustrate an adapter 8096 having structural, electrical and or functional features substantially identical and/or similar to those of the adapter 96 shown in FIGS. 14-18. In one embodiment, the adapter 8096 is removably received in a compartment 8180 provided in a side of an associated AED unit 8012. The adapter 8096 includes a pair of wire strips 8182 for electrically and/or mechanically connecting the adapter 8096 to the AED unit 8012. A locking mechanism (e.g., a latch mechanism) is provided for releasably locking the adapter 8096 in the compartment 8180. A release button 8184 is provided in the side of the AED unit 8012 for releasing the adapter 8096 from the compartment 8180 of the AED unit 8012.

FIGS. 40-42 illustrate an adapter 9096 having structural, electrical and or functional features substantially identical and/or similar to those of the adapter 96 shown in FIGS. 14-18. In one embodiment, the adapter 9096 is pivotally attached to a side of an associated AED unit 9012. In one embodiment, a locking mechanism (e.g., a latch mechanism) is provided for releasably locking the adapter 96 in a compartment 9180 formed in a side of the AED unit 9012.

FIG. 43 illustrates an adapter 10096 having structural, electrical and or functional features substantially identical and/or similar to those of the adapter 96 shown in FIGS. 14-18. In one embodiment, the adapter 10096 is removably placed in a compartment 10180 formed in a side of an associated AED unit 10012. The adapter 10096 includes a pair of opposing tabs 10186 for providing a grip for pulling the adapter 10096 from the compartment 10180.

It will be understood that the embodiments described herein are merely exemplary and that a person skilled in the art may make many variations and modifications without departing from the spirit and scope of the invention. All such variations and modifications are intended to be included within the scope of the invention.

We claim:

1. A resuscitation device, comprising a first resuscitation unit configured to perform a first resuscitation function, said first resuscitation unit having front and back sides; and a second resuscitation unit configured to perform a second resuscitation function, which is different from said first resuscitation function, said second resuscitation unit having front and rear sides, said first and second resuscitation units being configured so as to be assembled together as a single assembly such that said first and second resuscitation units can be carried conjointly for selectively performing at least one of said first and second functions.

2. The device of claim 1, wherein said first and second resuscitation units are configured to be arranged in a back-to-back fashion.

3. The device of claim 2, wherein said back side of said first resuscitation unit is configured to be removably attached to said back side of said second resuscitation unit.

4. The device of claim 1, wherein said first resuscitation unit includes a first handle; and wherein said second resuscitation unit includes a second handle, said first and second handles being configured such that when said first and second resuscitation units are assembled together, said first and second handles abut one another so as to form a single combined handle for conjointly carrying said first and second resuscitation units.

5. The device of claim 1, further comprising at least one storage pouch configured to be removably attached to one side of said first resuscitation unit and to one side of said second resuscitation unit when said first and second resuscitation units are assembled together as said single assembly.

6. The device of claim 5, wherein said at least one storage pouch is configured to inhibit said first and second resuscitation units from becoming detached from each other when said at least one storage pouch is attached to said one side of said first resuscitation unit and said one side of said second resuscitation unit.

7. The device of claim 1, further comprising a third resuscitation unit configured to perform a third resuscitation function different from said first and second resuscitation functions, each of said first, second and third resuscitation units being configured so as to be assembled together with another one of said first, second and third resuscitation units as a single assembly such that at least two of said first, second and third resuscitation units can be carried conjointly for selectively performing their respective ones of said first, second and third resuscitation functions.

8. The device of claim 1, wherein each of said first and second resuscitation units includes one of an automatic external defibrillator, a ventilator and an oxygenator.

9. The device of claim 8, wherein each of said first and second resuscitation units includes at least one connector configured for connection to an external device having a battery, each of said first and second resuscitation units containing circuitry for operating a corresponding one of said first and second resuscitation units via electricity received from the battery of the external device.

10. The device of claim 1, wherein each of said first and second resuscitation units includes a connector configured to be connected for connection to an external power supply including at least one of a DC power supply, an AC power supply and a solar power supply.

11. The device of claim 1, wherein at least one of said first and second resuscitation units includes a video conference feature which is equipped with a camera and a display panel mounted on a housing of a corresponding one of said first and second resuscitation units.

12. The device of claim 1, further comprising a basket having a first compartment configured to receive said first resuscitation unit and a second compartment configured to receive said second resuscitation unit.

13. The device of claim 12, wherein said basket includes at least one pouch for storing supplies and equipment.

14. The device of claim 1, wherein one of said first and second resuscitation units includes a bay; and wherein the other one of said first and second resuscitation units is configured to be received in said bay so as to form said single assembly.

15. The device of claim 1, wherein one of said first and second resuscitation units includes a ledge; and wherein the other one of said first and second resuscitation units is configured to be received on said ledge so as to form said single assembly.

16. A portable defibrillator system, comprising a portable defibrillator configured for delivering an electrical shock to a patient, said portable defibrillator including a first connector specifically configured for connection only with a first set of preconfigured electrode pad connectors; and an adaptor having a second connector, which is specifically configured for connection with said first connector of said defibrillator, and a plurality of third connectors, at least one of which is specifically configured for connection only with a second set of preconfigured electrode pad connectors, which are different from said first set of preconfigured electrode pad connectors, said second connector being electrically connected to said plurality of third connectors such that said first connector of said defibrillator can be electrically connected to said second set of preconfigured electrode pad connectors via said adaptor.

17. The portable defibrillator system of claim 16, wherein each of said plurality of third connectors is specifically configured for connection only with one set of preconfigured electrode pad connectors.

18. The portable defibrillator system of claim 16, wherein said portable defibrillator includes at least one connector configured for connection to an external device having a battery, said portable defibrillator including circuitry for operating said portable defibrillator via electricity received from the battery of the external device.

19. The portable defibrillator system of claim 16, wherein each of portable defibrillator includes a power connector configured for connection to an external power supply including at least one of a DC power supply, an AC power supply and a solar power supply.

20. An adaptor for a portable defibrillator configured for delivering an electrical shock to a patient, said portable defibrillator including a connector specifically configured for connection only with a first set of preconfigured electrode pad connectors, said adaptor comprising a first connector, which is specifically configured to connection with the connector of the defibrillator, and a plurality of second connectors, at least one of which is specifically configured for connection only with a second set of preconfigured electrode pad connectors, which are different from the first set of preconfigured electrode pad connectors, said first connector being electrically connected to said plurality of second connectors such that the connector of the defibrillator can be electrically connected to the second set of preconfigured electrode pad connectors via said adaptor.
